(19) United States
(12) Patent Application Publication
(10) Pub. No.: US 2025/0308064 A1
(43) Pub. Date: Oct. 2, 2025

(54) AUTOMATED DETECTION AND TRACKING OF A TWINKLING MARKER FROM COLOR DOPPLER VIDEO STREAM

(71) Applicant: Mayo Foundation for Medical Education and Research, Rochester, MN (US)

(72) Inventors: Matthew W. Urban, Rochester, MN (US); Christine U. Lee, Byron, MN (US); Luiz D. Vasconcelos, St. Paul, MN (US)

(21) Appl. No.: 19/096,022

(22) Filed: Mar. 31, 2025

Related U.S. Application Data
(60) Provisional application No. 63/571,622, filed on Mar. 29, 2024.

(51) Int. Cl. G06T 7/73; A61B 8/00; A61B 8/08; G06T 7/90

(52) U.S. Cl. CPC G06T 7/73; A61B 8/0833; A61B 8/46; A61B 8/488; A61B 8/5207; G06T 7/90; G06T 2207/10016; G06T 2207/10024; G06T 2207/10132; G06T 2207/30004; G06T 2207/30204

(57) ABSTRACT

A marker is localized using ultrasound imaging. Ultrasound video stream data are received with a computer system. The ultrasound video stream data are representative of ultrasound data acquired from a subject using an ultrasound system using Doppler imaging. Color image data are extracted from the ultrasound video stream data based on a color model. Marker position data are determined based on values of a color model component in the color image data. The marker position data are output using the computer system.

### BACKGROUND

Breast cancer is the most common cancer in women with 30-60% (depending on the defined group) metastasizing to axillary lymph nodes. The management of positive nodes is facilitated with the placement of markers, followed by neoadjuvant therapy and surgery. Ultrasound is the imaging modality of choice in the axilla, but currently available markers remain challenging to detect using conventional ultrasound imaging. When imaged under color Doppler ultrasound, some biopsy markers, localizers, or other implanted objects exhibit a twinkling artifact that facilitates their location by the radiologist or surgeon. There is a need for further improving their location and tracking.

### SUMMARY OF THE DISCLOSURE

The present disclosure addresses the aforementioned drawbacks by providing a method for localizing a marker using ultrasound imaging. Ultrasound video stream data are received with a computer system. The ultrasound video stream data are representative of ultrasound data acquired from a subject using an ultrasound system using Doppler imaging. Color image data are extracted from the ultrasound video stream data based on a color model. Marker position data are determined based on values of a color model component in the color image data. The marker position data are output using the computer system.

### BRIEF DESCRIPTION OF THE DRAWINGS

FIG. 1 is a flowchart of an example method for localizing and/or tracking a marker based on color model components in color image data extracted from video stream data representative of ultrasound data acquired with an ultrasound system.

FIG. 2 illustrates extracting color image data from ultrasound video stream data.

FIG. 3 illustrates generating a profile of a color model component along a spatial dimension (i.e., the x-axis in the illustrated example) of color image data.

FIG. 4 illustrates determining a position of a marker along a spatial dimension (i.e., the x-axis in the illustrated example) of color image data by convolving a function with the profile of the color model component along that spatial dimension.

FIG. 5 illustrates generating corrected color image data to remove false positive twinkling signals from the color image data.

FIG. 6 illustrates generating a profile of a color model component along another spatial dimension (i.e., the z-axis in the illustrated example) of corrected color image data.

FIG. 7 illustrates determining a position of a marker along another spatial dimension (i.e., the z-axis in the illustrated example) of corrected color image data by convolving a function with the profile of the color model component along that spatial dimension.

FIG. 8 illustrates generating an audio cue based on marker position data.

FIG. 9 is a block diagram of an example ultrasound system.

FIG. 10 is a block diagram of an example system for localizing and/or tracking a marker using ultrasound imaging.

FIG. 11 is a block diagram of example components that can implement that system of FIG. 10.

### DETAILED DESCRIPTION

Described here are systems and methods for imaging, localizing, and tracking twinkling markers (e.g., biopsy markers, localizers) in real-time using video stream data recorded from a Doppler ultrasound scanner. As a non-limiting example, ultrasound data can be analyzed on the ultrasound scanner or otherwise received from the ultrasound scanner and the color properties of the images in the ultrasound data can be used to locate the marker and track it in real time at more than 30 frames per second. An audio cue may be used to help the surgeon navigate to the marker. For example, the audio cue may include a pitch that is inversely proportional to the distance to the marker. Advantageously, the systems and methods described in the present disclosure are vendor agnostic. As another advantage, the systems and methods may be implemented using a standalone unit that can be located in an operating room setting.

In general, ultrasound data are received from the ultrasound sound scanner and the color Doppler images can be processed within the framework of a selected color model (e.g., an RGB color model) to localize and/or track a twinkling marker in real time based on properties of the color Doppler images associated with the selected color model. The twinkling marker may be a biopsy marker (e.g., placed at the time of biopsy), a localizer (e.g., placed at variable times and detectable by a surgeon), or the like. More generally, the marker may be any object that generates a suitable twinkling artifact signature that can be detected based on color Doppler images.

In some aspects, images from a color Doppler feed with both grayscale and color pixels overlaid on portions of the grayscale images are received from the ultrasound scanner. The standard deviation of the three RGB components (RGB-SD) of the images can be used to identify the location of an ultrasound marker based on the Doppler twinkling signals generated by the marker. Using the standard deviation of the three RGB components of the pixels has the advantage over processing grayscale pixels, which have low to zero standard deviation, whereas colored pixels have high standard deviation. The marker can be identified and localized in real-time above 30 frames per second.

In some other aspects, the HSV values of color Doppler images can be processed. Using the values of the HSV components of the pixels has the advantage that grayscale pixels have low to zero saturation-value (SV), whereas colored pixels have higher valued components.

The methods described in the present disclosure are able to track marker positions from ultrasound color Doppler video feed data, and therefore, have the potential for a vendor-agnostic implementation. Additionally, the acquired marker positions can be translated to an audio cue so users (e.g., ultrasound technicians, radiologists, surgeons, etc.) can track a marker position without visually relying on the ultrasound device monitor. Although reference is made to a single marker in some illustrative examples, it will be appreciated that multiple markers present in the imaging field-of-view may be detected using the systems and methods described in the present disclosure.

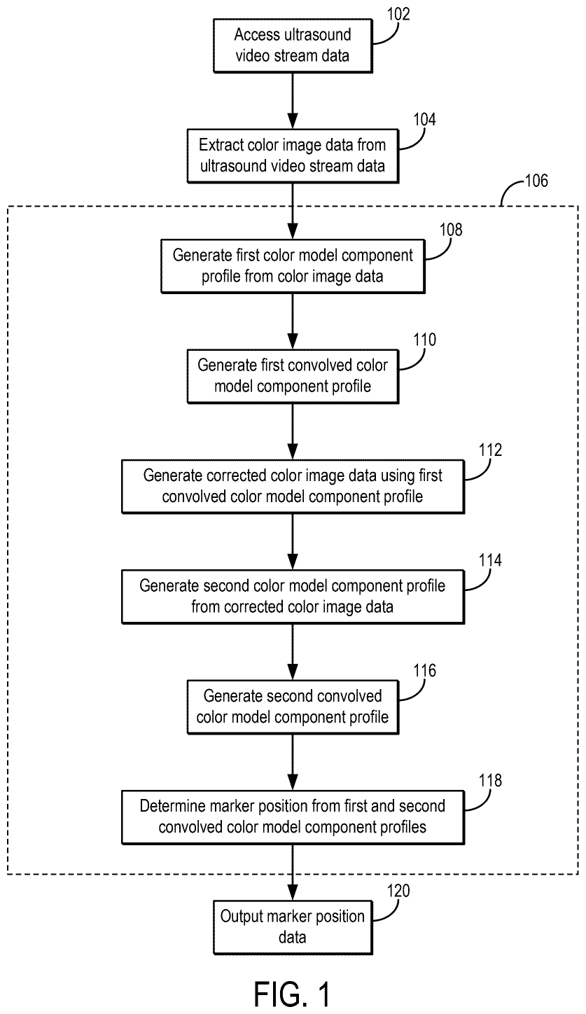

Referring now to FIG. 1, a flowchart is illustrated as setting forth the steps of an example method for localizing and/or tracking a marker using Doppler ultrasound.

The method includes accessing ultrasound video stream data with a computer system, as indicated at step 102. Accessing the ultrasound video stream data may include retrieving such data from a memory or other suitable data storage device or medium. Additionally or alternatively, accessing the ultrasound video stream data may include acquiring such data with an ultrasound system and transferring or otherwise communicating the data to the computer system, which may be a part of the ultrasound system.

In some instances, accessing the ultrasound video stream data includes extracting image frames from the ultrasound video stream data. In some other instances, accessing the ultrasound video stream data includes capturing or otherwise recording the ultrasound video stream data from a display of the ultrasound system or an associated computer system. For example, as images are displayed on the display of the ultrasound system, or the associated computer system, they can be captured, grabbed, or otherwise recorded as the ultrasound video stream data. In these instances, the ultrasound video stream data may not contain raw ultrasound data, but may instead represent image frames in a color space or color model that may be processed to determine the location of ultrasound markers in a vendor-agnostic manner. Accessing the ultrasound video stream data may, in some instances, can include interacting directly with the data produced by the ultrasound scanner (e.g., DICOM data) as opposed to duplicating images and working from a different set than the native images (e.g., by frame grabbing, screen capturing, or the like). In some cases, one or more regions-of-interest (ROIs) may be extracted or otherwise selected from the image frames in the ultrasound video steam data and the ROIs may be processed using the methods described below.

The ultrasound video stream data generally include color image data frames representative of color Doppler images, which may be stored as color images associated with a particular color space or color model. For example, the color image data frames may include images associated with a red-green-blue (RGB) color space or color model. In these instances, each pixel in the color images may be represented by a red channel component, a green channel component, and a blue channel component (i.e., RGB values). Additionally or alternatively, the color image data frames may be represented by other color spaces and/or color models. As an example, the color image data frames may include images associated with a hue-saturation-value (HSV) color space or color model. In these instances, each pixel in the color images may be represented by a hue component, a saturation component, and a value component (i.e., HSV values).

The ultrasound video stream data are generated from ultrasound data acquired from a subject in which at least one ultrasound marker has been positioned. The ultrasound marker includes any suitable marker that generates a twinkling artifact, signature, or signal in response to ultrasound transmitted to the region containing the marker.

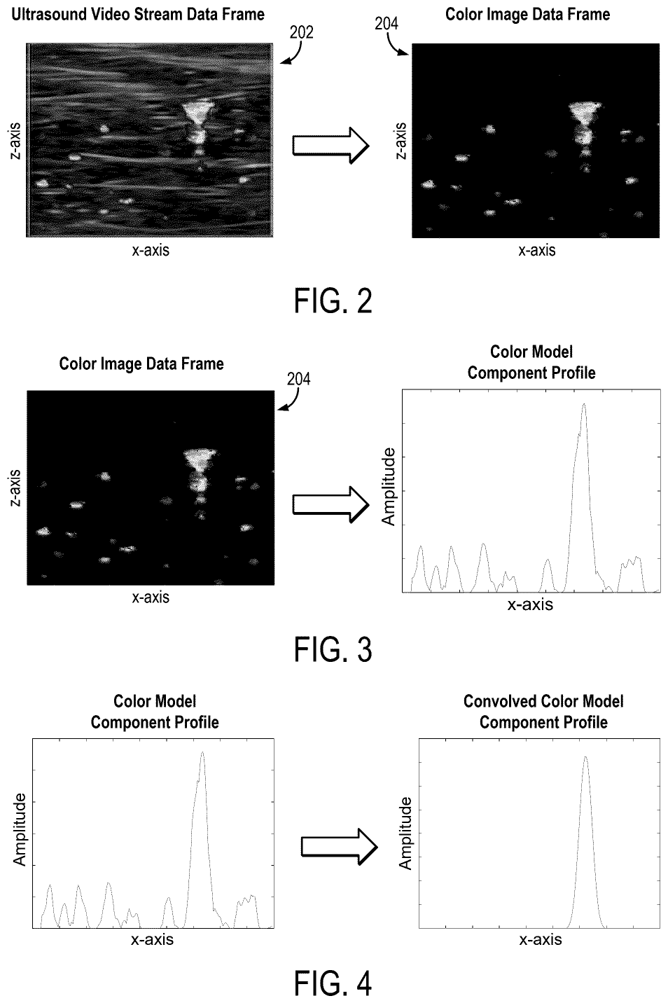

In some implementations, the ultrasound video stream data may also include grayscale images or other data representative of B-mode images, which may be stored as grayscale images. In these instances, the color Doppler image data can be overlaid on the grayscale B-mode image data. When the ultrasound video steam data comprise color images overlaid on grayscale images (e.g., color Doppler images overlaid on B-mode images), the color image data is first separated from the grayscale image data, as indicated at step 104. For example, a color model parameter filter can be applied to the ultrasound video stream data to separate the color image data from the grayscale image data. In general, the color model parameter filter separates the color image data from the grayscale image data based on properties of one or more parameters of a color model. As a non-limiting example, color image data may have a higher color model parameter value than grayscale image data. Based on these differences, the color model parameter filter can separate color image data from grayscale image data. FIG. 2 shows an example of filtering ultrasound video stream data 202 to separate color image data 204 from grayscale image data in the ultrasound video stream data 202.

As one non-limiting example, the color model parameter filter may be an RGB standard deviation (RGB-SD) filter that separates color image data and grayscale image data based on the standard deviation of RGB component values. An RGB pixel is composed of three integer values each with a range of 0-255. Grayscale pixels have the same or closely matched values of all three RGB values. As a result, the standard deviation of the three RGB components can be used to evaluate the “colorfulness” of a pixel. In this way, color image data can be separated from grayscale image data. Applying the RGB filter may thus include calculating the standard deviation of the RGB components and using a threshold to separate color image data from grayscale image data.

As another non-limiting example, the color model parameter filter may be a saturation-value (SV) filter that separates color image data and grayscale image data based on SV component values. An HSV pixel is composed of a hue component (e.g., an angle value around a color wheel, with values with a range of 0-360), a saturation component (e.g., a percentage value with a range of 0-100%), and a value component (e.g., a percentage value with a range of 0-100%). Grayscale pixels have low to zero SV components, whereas colored pixels have higher SV component values. In this way, color image data can be separated from grayscale image data based on the SV component values in the ultrasound video stream data (e.g., by comparing the SV component values to a threshold value).

The color image data from the ultrasound video stream data are then processed to localize and/or track a marker, as generally indicated by process block 106. In general, a color model component of the color image data is analyzed along a first spatial dimension of the color image data to determine the position of a probable marker along the second spatial dimension of the color image data, and the color image data is analyzed along the second spatial dimension to determine the position of a probable marker along the first spatial dimension. The first spatial dimension may be the depth dimension (e.g., the z-axis) of the color image data and the second spatial dimension may be the lateral, or horizontal, dimension (e.g., the x-axis) of the color image data.

First, a color model component value of the color image data is summed along the first spatial dimension of the images in the color image data to generate a color model component profile for the image, as indicated at step 108. As one example, the color model component may be an RGB-SD parameter. As another example, the color model component may be the value component of an HSV color model. In some cases, the color model component data may first be masked before summing along the first and second spatial dimensions. As a non-limiting example, when the color model component is an RGB-SD parameter, a binary mask may be retaining only standard deviation values above a threshold value, such as 0.1. The binary mask may then be applied to the color model component data to generate masked color model component data that are then summed along the first and second spatial dimensions.

FIG. 3 shows an example of summing a color model component in the color image data to generate a color model component profile. In the illustrated example, the color model component profile is a one-dimensional (1D) profile generated by summing the color model component value along the depth direction (i.e., the z-axis) of the color image data. Alternatively, the color model component value may be summed along the x-axis, the y-axis, or another spatial dimension of the color image data. For example, in some cases both a column-wise sum and a row-wise sum of the color model component data, which may be a 2D image, may be computed. In still other examples, the color model component profile may be a higher dimensional profile. For instance, when the color image data are three-dimensional (3D) data, the color model component profile may be a two-dimensional (2D) profile.

The color model component profile is then convolved with a function to identify the spatial position of the marker along the second spatial dimension of the color image data, as indicated at step 110. As a non-limiting example, the color model component profile may be convolved with a Gaussian function to identify the position of the marker along the second spatial dimension. For example, the position of the marker along the second spatial dimension can be identified based on a peak of the convolved color model profile. FIG. 4 shows an example of a Gaussian convolved color model component profile, illustrating the position of a probable marker along the x-axis. In some embodiments, more than one peak may be present in the convolved color model profile, indicating that more than one marker is present in the field-of-view of the ultrasound video stream data. In these instances, the position of the additional marker(s) may be similarly identified and recorded.

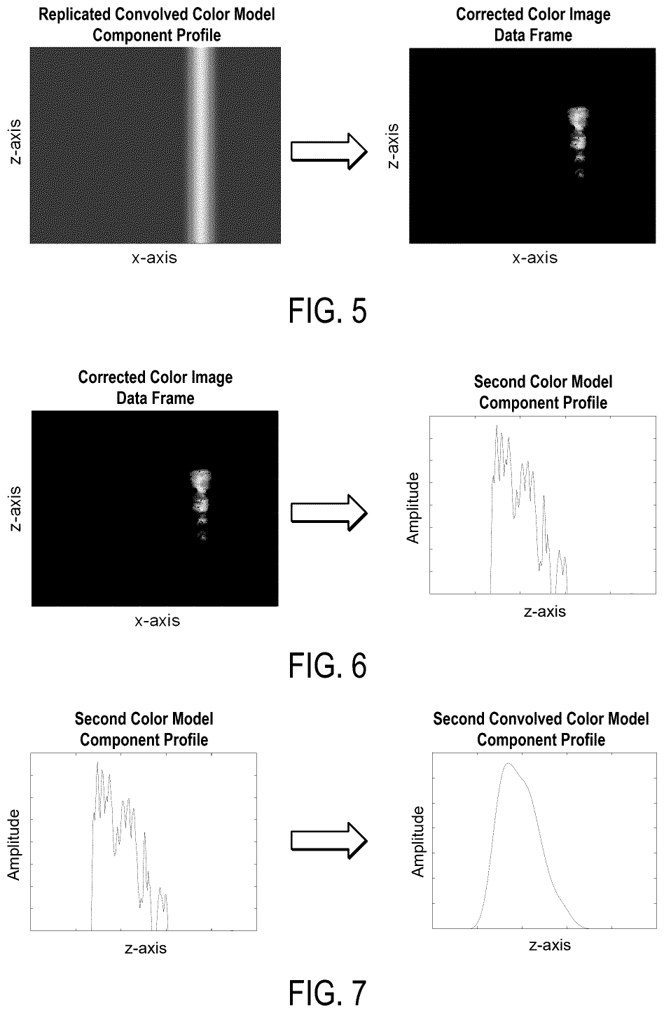

Corrected color image data are then generated by removing false positive artifacts (i.e., twinkling signals) from the color image data as indicated at step 112. For example, the convolved profile is replicated along the first spatial dimension (e.g., the z-axis) and then multiplied with the color image data to remove false positive twinkling signals. FIG. 5 shows an example of replicating a Gaussian convolved color model component profile along the z-axis and multiplying that with the color image data to remove false positive artifacts (i.e., false positive twinkling signals).

The color model component is then summed along the second spatial dimension (e.g., the x-axis) of the corrected color image data to generate a second color model component profile, as indicated at step 114. Like the first color model component profile, the second color model component profile represents a 1D profile of the color model component, but along the first spatial dimension rather than the second spatial dimension. FIG. 6 shows an example of summing a color model component in the corrected color image data along the second spatial dimension to generate a color model component profile. The second color model component profile is then convolved with a function (e.g., a Gaussian function, or the like) to generate a second convolved color model component profile, as indicated at step 116. The second convolved color model component profile identifies the spatial position of the probable marker along the first spatial dimension (e.g., the z-axis). For example, the position of the marker along the first spatial dimension can be identified based on a peak of the second convolved color model profile. FIG. 7 shows an example of a second convolved color model component profile, illustrating the position of a probable marker along the z-axis. In some embodiments, more than one peak may be present in the second convolved color model profile, indicating that more than one marker is present in the field-of-view of the ultrasound video stream data. In these instances, the position of the additional marker(s) may be similarly identified and recorded.

The position of each probable marker in the color image data is thus determined based on the first and second convolved color model component profiles and stored as marker position data, as indicated at step 118. Additionally or alternatively, a binary mask indicating the probably location of the marker may be generated and smoothed based on the color model component profiles.

The marker position data may then be output using the computer system, as indicated at step 120. As one example, outputting the marker position data can include displaying the marker position data to a user via the computer system. For instance, the location of a marker can be displayed as an overlay on the ultrasound video stream data. By way of example, when the marker position data include a binary mask indicating the marker position, the binary mask may be used to generate a visual indication of the probable marker position on the ultrasound video stream data.

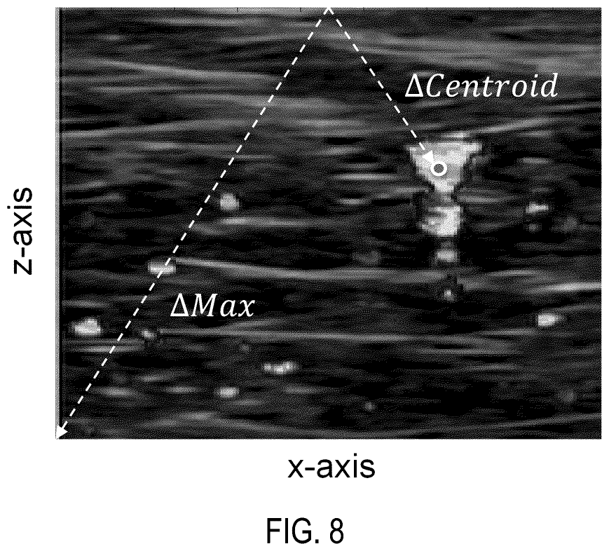

As mentioned above, in some implementations outputting the marker position data may include generating an audio cue based on the marker position data and outputting the audio cue via a speaker, or the like. FIG. 8 illustrates an example process for determining the parameters for an audio cue based on the marker position data. A centroid of the marker is computed from the marker position data, and the distance, Δcentroid, from a location in the ultrasound video stream data to the marker centroid is computed. As illustrated, the distance from the top-center of the ultrasound video stream data frame to the marker centroid is computed. The maximum distance, Δmax, in the ultrasound video stream data frame is also computed. The maximum distance, Δmax, is then compared with the centroid distance, Δcentroid, to generate parameters of the audio cue. As one non-limiting example, the audio cue pitch can be defined as,

$$200 Hz + 800 Hz \cdot ( 1 - \frac{Δ centroid}{Δ max} ) .$$

In other examples, the frequency may be defined as a function of Δcentroid and Δmax. The repetition frequency of a tone could also be used and modified to provide the audio cue (e.g., faster repetition indicating a closer proximity to the marker, etc.).

The implementation of generating an audio cue based on the marker position data can enable a surgeon, or other user, to track the position or a marker without visually relying on the ultrasound device monitor.

In an example study evaluating the systems and methods described in the present disclosure, a polymethyl methacrylate (PMMA) marker was placed 17 mm deep into a polyvinyl alcohol cryogel phantom. An ultrasound system with a linear array transducer was used to acquire Doppler images from the phantom inside a water tank. The duplex images (B-mode with color Doppler) were acquired with a framegrabber module from the HDMI output from the scanner and captured using MATLAB. The standard deviation of the three RGB components (RGB-SD) was used to identify the Doppler twinkling location. Colored pixels have high RGB-SD values, whereas grayscale pixels have low to zero RGB-SD, as all three components have similar values. To detect the occurrence of a significant twinkling artifact the RGB standard deviation was summed vertically, and its mean and overall standard deviation were compared. True positive occurrences tend to present high mean values for RGB-SD, but low standard deviation, due to the concentration of color intensity at a small region. After detection, the x-position (horizontal direction) was acquired by identifying the peak of the artifact standard deviation sum. The image was then multiplied with a vertical Gaussian function, isolating the artifact from false positives horizontally. Finally, the z-position (vertical direction) was acquired by repeating the sum and Gaussian convolution steps horizontally.

To evaluate the method's tracking performance, the transducer was placed on a high-precision translation stage, and its acquisition position was altered 1.0 mm vertically, and, subsequently, 3.5 mm horizontally, both in increments of 0.10 mm, below the image spatial resolution of 0.13 mm/pixel. The accuracy was calculated by the mean absolute error between the estimated and real displacement recorded using the translational stage. The precision was calculated by the spatial standard deviation throughout all the acquisitions at each given position. The algorithm successfully localized the twinkling marker with an accuracy of 0.145 mm, and a precision of 0.082-0.414 mm (mean: 0.138 mm in x, and 0.229 mm in z). The method was able to identify and localize the marker in real-time above 30 frames per second.

The proposed method was able to track marker position from ultrasound color Doppler video feed and, therefore, have the potential for vendor-agnostic implementation. The tracking performance was primarily limited by the ultrasound image feed resolution, with accuracy of 0.145 mm and precision between 0.082 and 0.414 mm. Additionally, the acquired marker position will be translated to an audio cue so radiologists or surgeons can track the marker position without visually relying on the ultrasound device monitor.

In another example study evaluating the systems and methods described in the present disclosure, a PMMA marker was placed 17 mm deep into a polyvinyl alcohol cryogel phantom. An ultrasound system with a linear array transducer was used to acquire Doppler images from the phantom inside a water tank. The duplex images (B-mode with color Doppler) were acquired with a framegrabber module from the HDMI output from the scanner and captured using MATLAB. The Hue-Saturation-Value (HSV) components were used to identify the Doppler twinkling location. Grayscale pixels have low to zero Saturation-Value (SV) whereas colored pixels have higher components. The algorithm applied an SV filter, summed the Value component along the z-axis and the x-axis, as described above, to find peaks given an arbitrary threshold. The z-position and x-position of the twinkling centroid was returned and depicted as a small red circle overlaid on the original image.

To evaluate the method's tracking performance, the transducer position was altered in increments of 0.10 mm, below the image spatial resolution of 0.13 mm/pixel in both z and x dimensions. The algorithm successfully localized the twinkling marker with an accuracy of 0.087 mm mean absolute error, and a precision with a spatial standard deviation of 0.101-0.465 mm (mean: 0.163 mm in x, and 0.268 mm in z). The method was able to localize the marker in real-time above 30 frames per second.

An audio cue was composed by a 0.05 s tone activated every two frames. The pitch frequency was inversely proportional to the Euclidian distance of the centroid to the frame top-center, within a 200 to 1000 Hz range. By incorporating audio feedback, a surgeon or other user is advantageously provided with a tool to monitor marker position without dependence on visual cues from the ultrasound device monitor.

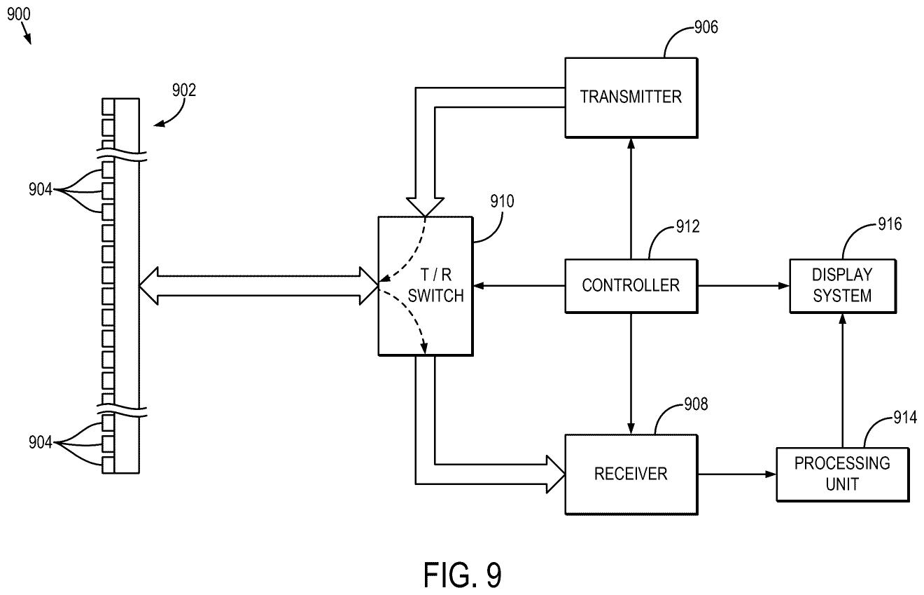

FIG. 9 illustrates an example of an ultrasound system 900 that can implement the methods described in the present disclosure. The ultrasound system 900 includes a transducer array 902 that includes a plurality of separately driven transducer elements 904. The transducer array 902 can include any suitable ultrasound transducer array, including linear arrays, curved arrays, phased arrays, and so on. Similarly, the transducer array 902 can include a 1D transducer, a 1.5D transducer, a 1.75D transducer, a 2D transducer, a 3D transducer, and so on.

When energized by a transmitter 906, a given transducer element 904 produces a burst of ultrasonic energy. The ultrasonic energy reflected back to the transducer array 902 (e.g., an echo) from the object or subject under study is converted to an electrical signal (e.g., an echo signal) by each transducer element 904 and can be applied separately to a receiver 908 through a set of switches 910. The transmitter 906, receiver 908, and switches 910 are operated under the control of a controller 912, which may include one or more processors. As one example, the controller 912 can include a computer system.

The transmitter 906 can be programmed to transmit unfocused or focused ultrasound waves. In some configurations, the transmitter 906 can also be programmed to transmit diverged waves, spherical waves, cylindrical waves, plane waves, or combinations thereof. Furthermore, the transmitter 906 can be programmed to transmit spatially or temporally encoded pulses.

The receiver 908 can be programmed to implement a suitable detection sequence for the imaging task at hand. In some embodiments, the detection sequence can include one or more of line-by-line scanning, compounding plane wave imaging, synthetic aperture imaging, and compounding diverging beam imaging.

In some configurations, the transmitter 906 and the receiver 908 can be programmed to implement a high frame rate. For instance, a frame rate associated with an acquisition pulse repetition frequency (“PRF”) of at least 100 Hz can be implemented. In some configurations, the ultrasound system 900 can sample and store at least one hundred ensembles of echo signals in the temporal direction.

A scan can be performed by setting the switches 910 to their transmit position, thereby directing the transmitter 906 to be turned on momentarily to energize transducer elements 904 during a single transmission event according to a selected imaging sequence. The switches 910 can then be set to their receive position and the subsequent echo signals produced by the transducer elements 904 in response to one or more detected echoes are measured and applied to the receiver 908. The separate echo signals from the transducer elements 904 can be combined in the receiver 908 to produce a single echo signal.

The echo signals are communicated to a processing unit 914, which may be implemented by a hardware processor and memory, to process echo signals or images generated from echo signals. In some instances, the processing unit 914 may implement Doppler processing (e.g., color Doppler, other Doppler modes) of ultrasound data. As an example, the processing unit 914 can localize and/or track twinkling ultrasound markers using the methods described in the present disclosure. Images produced from the echo signals by the processing unit 914 can be displayed on a display system 916. As described above, images displayed on the display system can be captured or otherwise recorded as video stream data that can be processed using the methods described in the present disclosure to localize and/or track an ultrasound marker.

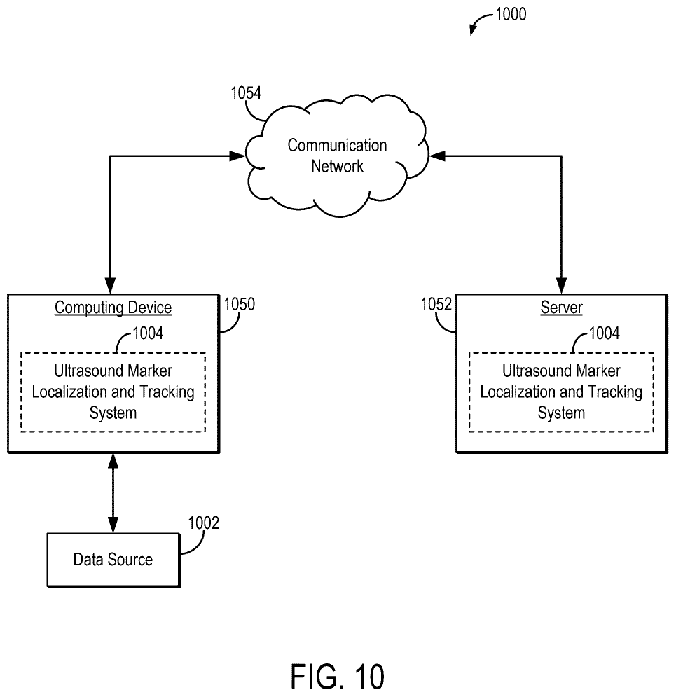

FIG. 10 shows an example of a system 1000 for localizing and/or tracking twinkling ultrasound markers in accordance with some embodiments described in the present disclosure. As shown in FIG. 10, a computing device 1050 can receive one or more types of data (e.g., ultrasound video stream data, color image data) from data source 1002. In some embodiments, computing device 1050 can execute at least a portion of an ultrasound marker localization and tracking system 1004 to localize and/or track one or more ultrasound markers from data received from the data source 1002.

Additionally or alternatively, in some embodiments, the computing device 1050 can communicate information about data received from the data source 1002 to a server 1052 over a communication network 1054, which can execute at least a portion of the ultrasound marker localization and tracking system 1004. In such embodiments, the server 1052 can return information to the computing device 1050 (and/or any other suitable computing device) indicative of an output of the ultrasound marker localization and tracking system 1004.

In some embodiments, computing device 1050 and/or server 1052 can be any suitable computing device or combination of devices, such as a desktop computer, a laptop computer, a smartphone, a tablet computer, a wearable computer, a server computer, a virtual machine being executed by a physical computing device, and so on. In some cases, the computing device 1050 may be part of the ultrasound scanner used to collect ultrasound video stream data. In these instances, the ultrasound video stream data may be analyzed on the ultrasound system itself. For example, one or more processors of the ultrasound system (e.g., processing unit 914 of ultrasound system 900) may implement the computing device 1050. In still other cases, the computing device 1050 may be separate from the ultrasound system, such that the ultrasound video stream data are processed offline relative to the ultrasound system. The computing device 1050 and/or server 1052 can also reconstruct images from the data.

In some embodiments, data source 1002 can be any suitable source of data (e.g., measurement data, images reconstructed from measurement data, processed image data, video stream data), such as an ultrasound system, another computing device (e.g., a server storing measurement data, images reconstructed from measurement data, processed image data, video stream data), and so on. In some embodiments, data source 1002 can be local to computing device 1050. For example, data source 1002 can be incorporated with computing device 1050 (e.g., computing device 1050 can be configured as part of a device for measuring, recording, estimating, acquiring, or otherwise collecting or storing data). As another example, data source 1002 can be connected to computing device 1050 by a cable, a direct wireless link, and so on. Additionally or alternatively, in some embodiments, data source 1002 can be located locally and/or remotely from computing device 1050, and can communicate data to computing device 1050 (and/or server 1052) via a communication network (e.g., communication network 1054).

In some embodiments, communication network 1054 can be any suitable communication network or combination of communication networks. For example, communication network 1054 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 1054 can be a local area network, a wide area network, a public network (e.g., the Internet), a private or semi-private network (e.g., a corporate or university intranet), any other suitable type of network, or any suitable combination of networks. Communications links shown in FIG. 10 can each be any suitable communications link or combination of communications links, such as wired links, fiber optic links, Wi-Fi links, Bluetooth links, cellular links, and so on.

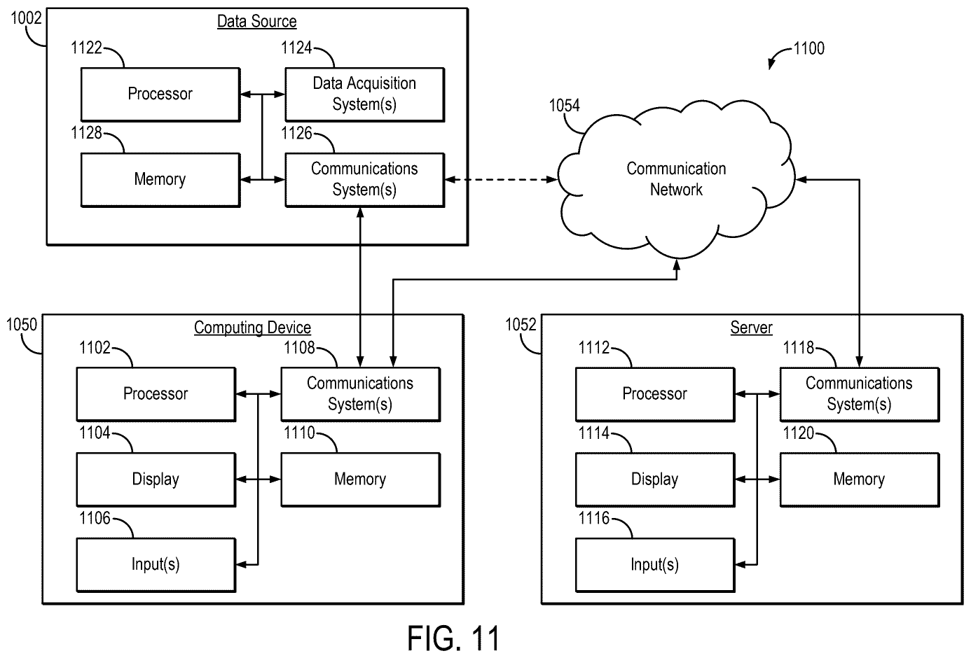

Referring now to FIG. 11, an example of hardware 1100 that can be used to implement data source 1002, computing device 1050, and server 1052 in accordance with some embodiments of the systems and methods described in the present disclosure is shown.

As shown in FIG. 11, in some embodiments, computing device 1050 can include a processor 1102, a display 1104, one or more inputs 1106, one or more communication systems 1108, and/or memory 1110. In some embodiments, processor 1102 can be any suitable hardware processor or combination of processors, such as a central processing unit (“CPU”), a graphics processing unit (“GPU”), and so on. In some embodiments, display 1104 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 1106 can include any suitable input devices and/or sensors that can be used to receive user input, such as a keyboard, a mouse, a touchscreen, a microphone, and so on.

In some embodiments, communications systems 1108 can include any suitable hardware, firmware, and/or software for communicating information over communication network 1054 and/or any other suitable communication networks. For example, communications systems 1108 can include one or more transceivers, one or more communication chips and/or chip sets, and so on. In a more particular example, communications systems 1108 can include hardware, firmware, and/or software that can be used to establish a Wi-Fi connection, a Bluetooth connection, a cellular connection, an Ethernet connection, and so on.

In some embodiments, memory 1110 can include any suitable storage device or devices that can be used to store instructions, values, data, or the like, that can be used, for example, by processor 1102 to present content using display 1104, to communicate with server 1052 via communications system(s) 1108, and so on. Memory 1110 can include any suitable volatile memory, non-volatile memory, storage, or any suitable combination thereof. For example, memory 1110 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 1110 can have encoded thereon, or otherwise stored therein, a computer program for controlling operation of computing device 1050. In such embodiments, processor 1102 can execute at least a portion of the computer program to present content (e.g., images, user interfaces, graphics, tables), receive content from server 1052, transmit information to server 1052, and so on. For example, the processor 1102 and the memory 1110 can be configured to perform the methods described herein (e.g., the method of FIG. 1).

In some embodiments, server 1052 can include a processor 1112, a display 1114, one or more inputs 1116, one or more communications systems 1118, and/or memory 1120. In some embodiments, processor 1112 can be any suitable hardware processor or combination of processors, such as a CPU, a GPU, and so on. In some embodiments, display 1114 can include any suitable display devices, such as an LCD screen, LED display, OLED display, electrophoretic display, a computer monitor, a touchscreen, a television, and so on. In some embodiments, inputs 1116 can include any suitable input devices and/or sensors that can be used to receive user input, such as a keyboard, a mouse, a touchscreen, a microphone, and so on.

In some embodiments, communications systems 1118 can include any suitable hardware, firmware, and/or software for communicating information over communication network 1054 and/or any other suitable communication networks. For example, communications systems 1118 can include one or more transceivers, one or more communication chips and/or chip sets, and so on. In a more particular example, communications systems 1118 can include hardware, firmware, and/or software that can be used to establish a Wi-Fi connection, a Bluetooth connection, a cellular connection, an Ethernet connection, and so on.

In some embodiments, memory 1120 can include any suitable storage device or devices that can be used to store instructions, values, data, or the like, that can be used, for example, by processor 1112 to present content using display 1114, to communicate with one or more computing devices 1050, and so on. Memory 1120 can include any suitable volatile memory, non-volatile memory, storage, or any suitable combination thereof. For example, memory 1120 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1120 can have encoded thereon a server program for controlling operation of server 1052. In such embodiments, processor 1112 can execute at least a portion of the server program to transmit information and/or content (e.g., data, images, a user interface) to one or more computing devices 1050, receive information and/or content from one or more computing devices 1050, receive instructions from one or more devices (e.g., a personal computer, a laptop computer, a tablet computer, a smartphone), and so on.

In some embodiments, the server 1052 is configured to perform the methods described in the present disclosure. For example, the processor 1112 and memory 1120 can be configured to perform the methods described herein (e.g., the method of FIG. 1).

In some embodiments, data source 1002 can include a processor 1122, one or more data acquisition systems 1124, one or more communications systems 1126, and/or memory 1128. In some embodiments, processor 1122 can be any suitable hardware processor or combination of processors, such as a CPU, a GPU, and so on. In some embodiments, the one or more data acquisition systems 1124 are generally configured to acquire data, images, or both, and can include an ultrasound system. Additionally or alternatively, in some embodiments, the one or more data acquisition systems 1124 can include any suitable hardware, firmware, and/or software for coupling to and/or controlling operations of an ultrasound system. In some embodiments, one or more portions of the data acquisition system(s) 1124 can be removable and/or replaceable.

Note that, although not shown, data source 1002 can include any suitable inputs and/or outputs. For example, data source 1002 can include input devices and/or sensors that can be used to receive user input, such as a keyboard, a mouse, a touchscreen, a microphone, a trackpad, a trackball, and so on. As another example, data source 1002 can include any suitable display devices, such as an LCD screen, an LED display, an OLED display, an electrophoretic display, a computer monitor, a touchscreen, a television, etc., one or more speakers, and so on.

In some embodiments, communications systems 1126 can include any suitable hardware, firmware, and/or software for communicating information to computing device 1050 (and, in some embodiments, over communication network 1054 and/or any other suitable communication networks). For example, communications systems 1126 can include one or more transceivers, one or more communication chips and/or chip sets, and so on. In a more particular example, communications systems 1126 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1128 can include any suitable storage device or devices that can be used to store instructions, values, data, or the like, that can be used, for example, by processor 1122 to control the one or more data acquisition systems 1124, and/or receive data from the one or more data acquisition systems 1124; to generate images from data; present content (e.g., data, images, a user interface) using a display; communicate with one or more computing devices 1050; and so on. Memory 1128 can include any suitable volatile memory, non-volatile memory, storage, or any suitable combination thereof. For example, memory 1128 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1128 can have encoded thereon, or otherwise stored therein, a program for controlling operation of data source 1002. In such embodiments, processor 1122 can execute at least a portion of the program to generate images, transmit information and/or content (e.g., data, images, a user interface) to one or more computing devices 1050, receive information and/or content from one or more computing devices 1050, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for localizing a marker using ultrasound imaging, the method comprising:
receiving ultrasound video stream data with a computer system, the ultrasound video stream data being representative of ultrasound data acquired from a subject using an ultrasound system using Doppler imaging;
extracting, using the computer system, color image data from the ultrasound video stream data based on a color model;
determining, using the computer system, marker position data based on values of a color model component in the color image data; and
outputting the marker position data using the computer system, wherein the marker position data indicate the location of the marker in the subject.

2. The method of claim 1, wherein the color model is a red-green-blue (RGB) color model.

3. The method of claim 2, wherein the color model component comprises a standard deviation of RGB components in the color image data.

4. The method of claim 2, wherein the color image data are extracted from the ultrasound video stream data based on a standard deviation of RGB components in the ultrasound video stream data.

5. The method of claim 1, wherein the color model is a hue-saturation-value color model.

6. The method of claim 5, wherein the color model component comprises a value component of the color image data.

7. The method of claim 5, wherein the color image data are extracted from the ultrasound video stream data based on saturation-value (SV) components in the ultrasound video stream data.

8. The method of claim 1, wherein the marker position data comprise a first position of the marker along a first spatial dimension and a second position of the marker along a second spatial dimension.

9. The method of claim 8, wherein the second position of the marker is determined by:
generating a first color model component profile by summing values of the color model component along the first spatial dimension for each position of the second spatial dimension in the color image data; and
determining the second position of the marker based on the first color model component profile.

10. The method of claim 9, wherein determining the second position of the marker based on the first color model component profile comprises:
convolving the first color model component profile with a function to generate a first convolved color model component profile; and
determining the second position of the marker based on a peak of the first convolved color model component profile.

11. The method of claim 8, wherein the first position of the marker is determined by:
generating corrected color image data from the color image data, wherein the corrected color image data have been corrected to remove false positive twinkling signals;
generating a second color model component profile by summing values of the color model component along the second spatial dimension for each position of the first spatial dimension in the corrected color image data; and
determining the first position of the marker based on the second color model component profile.

12. The method of claim 11, wherein determining the first position of the marker based on the second color model component profile comprises:
convolving the second color model component profile with a function to generate a second convolved color model component profile; and
determining the first position of the marker based on a peak of the second convolved color model component profile.

13. The method of claim 11, wherein the corrected color image data are generated by multiplying the color image data by a convolved color component profile associated with the second spatial dimension and replicated along the first spatial dimension.

14. The method of claim 13, wherein the convolved color component profile associated with the second spatial dimension is generated by:
generating a first color model component profile by summing values of the color model component along the first spatial dimension for each position of the second spatial dimension in the color image data; and
convolving the first color model component profile with a function to generate the convolved color component profile associated with the second spatial dimension.

15. The method of claim 8, wherein the first spatial dimension is a depth dimension and the second spatial dimension is a lateral dimension.

16. The method of claim 1, wherein outputting the marker position data using the computer system comprises generating a visual display element based on the marker position data and displaying the visual display element on the ultrasound video stream data.

17. The method of claim 1, wherein outputting the marker position data using the computer system comprises determining audio cue parameters based on the marker position data and generating an audio cue with the computer system using the audio cue parameters.

18. The method of claim 17, wherein the audio cue parameters include a pitch of the audio cue.

19. The method of claim 18, wherein the pitch of the audio cue is determined based on a centroid distance of the marker position data compared to a maximum distance in the color image data.

20. The method of claim 1, wherein a plurality of markers is located within the subject and the marker position data indicate the location of each of the plurality of markers in the subject.

21. The method of claim 1, wherein the computer system comprises a part of the ultrasound system.

22. The method of claim 1, wherein the computer system is remote to the ultrasound system.
